(19) United States
(12) Patent Application Publication
(10) Pub. No.: US 2007/0207213 A1
(43) Pub. Date: Sep. 6, 2007

(54) Injectable Microspheres From Unsaturated Functionalized Polyhydric Alcohol Esters

(75) Inventors: Chih-Chang Chu, Ithaca, NY (US); Da Qing Wu, Richmond, VA (US)

Correspondence Address: BACON & THOMAS, PLLC, 625 SLATERS LANE, FOURTH FLOOR, ALEXANDRIA, VA, 22314, US

(73) Assignee: Cornell Research Foundation, Inc., Ithaca, NY (US)

(21) Appl. No.: 11/628,650

(22) PCT Filed: Jun. 16, 2005
(86) PCT No.: PCT/US05/21110

§ 371 Date: Dec. 6, 2006

Related U.S. Application Data
(60) Provisional application No. 60/582,823, filed on Jun. 28, 2004.

(51) Int. Cl. A61K 9/50; B01J 13/04

(52) U.S. Cl. 424/490; 264/4.1

(57) ABSTRACT

Injectable drug laden microspheres are obtained from unsaturated functionalized polyhydric alcohol ester of polyester by a method comprising dissolving the unsaturated functionalized ester in a hydrophobic organic solvent, dissolving drug and/or biologically active agent in water, admixing the two solutions to form water-in-oil emulsion, forming an aqueous solution of stabilizer, admixing the water-in-oil emulsion and the stabilizer solution to form water-in-oil-in-water emulsion, and evaporating the organic solvent.

### CROSS-REFERENCE TO RELATED APPLICATIONS

This application claims the benefit of U.S. Provisional Patent Application No. 60/582,823, filed Jun. 28, 2004, the whole of which is incorporated herein by reference.

The invention was made at least in part with U.S. Government support under National Textile Center Grant No. 00-27-07400 which is provided by the U.S. Department of Commerce. The United States Government has certain rights in the invention.

### TECHNICAL FIELD

This invention is directed at a method for forming a drug/biologically active agent laden injectable hydrogel microsphere and a drug/biologically active agent laden microsphere formed thereby which is useful for controlled release of drug/biologically active agent in the body.

### BACKGROUND OF THE INVENTION

Microspheres with encapsulated or covalently bonded drug allow provision of an injectable suspension as a substitute for surgical implantation and allow administration of multiple drugs in a single injection. These microspheres provide an initial burst to reach a therapeutic concentration followed by a zero-order release of drug to maintain the therapeutic level by compensating for metabolic loss. The microspheres thus provide a sustained release therapeutic concentration.

Microspheres of biodegradable polyesters from D,L-lactide/glycolide and microspheres of biodegradable polyesters from ε-caprolactone have received attention for controlling release in the body of pharmaceutical agents and macromolecules. However, these polyesters are relatively hydrophobic and a more hydrophilic surface is desirable on an injectable microsphere to increase effective lifetime in the circulatory system and to reduce the occurrence of inflammatory response. Hydrophilic characteristics have been achieved by surface modification of the polyester microspheres with hydrophilic polymers. Polyester microspheres with more hydrophilic surfaces have not heretofore been obtained without relying on chemical attachment or physical absorption of hydrophilic polymers.

### SUMMARY OF THE INVENTION

It has been discovered herein that biodegradable injectable polyester microspheres with surface hydrophilicity can be prepared without the requirements of surface modification with hydrophilic polymer by forming the microspheres from unsaturated functionalized, e.g., double bond functionalized, polyhydric alcohol ester of polyester, e.g., those described in U.S. Pat. No. 6,592,895 and Lang, M., et al., Journal of Polymer Science: Part A: Polymer Chemistry, Vol. 40, 1127-1141 (2002), the whole of both of which are incorporated herein by reference.

In one embodiment herein, denoted the first embodiment, the invention is directed at forming a drug/biologically active agent laden biodegradable injectable microsphere and relies on a double emulsion technique and comprises the steps of:

(a) dissolving unsaturated functionalized, e.g., double bond functionalized, polyhydric alcohol ester of polyester in hydrophobic organic solvent,

(b) dissolving water soluble drug and/or other biologically active agent in water,

(c) admixing the solutions formed in step (a) and step (b) to form a first emulsion where the solution formed in step (a) constitutes continuous phase and the solution formed in step (b) constitutes the disperse phase,

(d) dissolving stabilizer in water,

(e) admixing the solution formed in step (d) with the emulsion formed in step (c) to form a water-in-oil-in-water emulsion where solution of step (d) constitutes the continuous phase and the emulsion formed in step (c) constitutes the disperse phase, (f) evaporating the organic solvent from water-in-oil-in-water emulsion formed in step (e), to form hardened microspheres from the unsaturated functionalized polyhydric alcohol ester of polyester, encapsulating said drug and/or other biologically active agent,

(g) recovering the microspheres with drug and/or other biologically active agent encapsulated therein.

In another case, denoted the second embodiment, the drug and/or other biologically active agent is capable of reacting to covalently bond to the unsaturated functionality and the hardened microspheres formed in step (f) and recovered in step (g) are reacted at said unsaturated functionality to covalently bond to the microsphere.

Preferably, the unsaturated functionalized, e.g., the double bond functionalized, polyhydric alcohol esters for the first and second embodiments are obtained by polymerizing ε-caprolactone monomer or a blend of ε-caprolactone and lactide monomer or glycolide monomer in the presence of a polyhydric alcohol containing from 3 to 6 hydroxyl groups to form polyhydric alcohol ester where the acyl groups contain free hydroxyl as their terminal ends (PGCL) and reacting with maleic anhydride to convert some or each of the free hydroxyls to moiety containing 2-carboxy ethenyl group, particularly 1-carboxyl-2-carboxy ethenyl to form maleic acid ester of (PGCL) which is termed (PGCLM).

The polyhydric alcohol esters of polyesters normally have three different kinds of functional terminal groups, namely unreacted —OH, —COOH and >C═C< and are plural arm, e.g., three-arm compounds.

The evaporation of solvent in step (f) of the method of the first embodiment causes hardening of the microsphere by polymer precipitation.

In another embodiment herein, denoted the third embodiment of the invention, the invention is directed at a biodegradable injectable microsphere having a mean transverse dimension ranging from 15 to 60 μm, formed of hardened unsaturated functionalized, e.g., double bond functionalized, plural arm polyhydric alcohol ester of polyester, for example, loaded with from 1 to 10% by weight of the microsphere of a drug or other biologically active agent, e.g., a protein, for sustained release after injection of the microsphere over a period ranging to a few months. The unsaturated functionality allows covalent bonding to biologically active agents for delayed release, as well as, provides the opportunity to form hydrogel at microsphere surface with the advantage of allowing two different release modes, one from within the microsphere and the other from within the hydrogel. In a preferred case, the microsphere is formed of hardened double bond functionalized polyhydric alcohol ester of polyester obtained by polymerizing ε-caprolactone monomer in the presence of glycerol to form polyhydric alcohol ester where the acyl groups contain free hydroxyl at their terminal ends and reacting with maleic anhydride to convert some or each of the free hydroxyls to moiety containing carboxy ethenyl group, particularly 1-carboxyl-2-carboxy ethenyl terminal group; that is, the microsphere is formed of double bond functionalized polyhydric alcohol ester of polyester which is terminal functionalized three-arm poly(ε-caprolactone) maleic acid having —OH, —COOH and >C═C< functional groups. In an alternative, the surface of the microsphere has been converted to a hydrogel, e.g., by crosslinking at surface double bonds. Loading can be obtained by including, e.g., encapsulating, drug or other biologically active agent, e.g., any water soluble peptides or proteins, or water-soluble vitamins (including all vitamins B, biotin, folic acid and ascorbic acid) within the microsphere and/or within hydrogel at microsphere surface and/or by covalent bonding to functional group(s) of the microsphere (i.e., via terminal —OH, —COOH and >C═C< functional groups). Drug or other biologically active agents for covalent bonding to functional group(s) of the microsphere include, for example, eneynes (anti-cancerous dyes) that are compounds that contain both a carbon-carbon double bond (ene) and a carbon-carbon triple bond (yne). The microspheres herein have a hydrophilic surface and have a longer lifetime in the circulatory system than microspheres with a hydrophobic surface, for better and more sustainable delivery of therapeutic agent.

The term “biodegradable” is used herein to mean capable of being broken down by various enzymes such as trypsins, lipases and lysomes in the normal functioning of the human body and living organisms (e.g., bacteria) and/or water environment.

The molecular weights and polydispersities herein, are determined by gel permeation chromatography using polystyrene standards. More particularly molecular weights of prepared polymers (Mₙ) and M_{w}) are determined by gel permeation chromatography (GPC) using tetrahydrofuran (THF) as eluant (1.0 ml/min) with a Water 510 HPLC pump, a Water U6K injector, three PSS SDV columns (linear and 10⁴ and 100 angstroms) in series, and a Milton ROM differential refractometer, and the sample concentration is 5-10 mg/ml of THF and the columns are calibrated by polystyrene standards having a narrow molecular weight distribution.

### BRIEF DESCRIPTION OF THE DRAWINGS

FIG. 1 shows chemical structures for PGCL and PGCLM and a representative structure of NPGCLM;

FIG. 2A shows size distribution for PGCLM65 microspheres formed in the working example;

FIG. 2B shows size distribution for PGCLM81 microspheres formed in the working example;

FIG. 2C shows size distribution for PGCLM61 microspheres formed in the working example;

FIG. 3A shows cumulative release of OVA for PGCLM41, PGCLM61, PGCLM81 and PGCLM85 obtained in the working example;

FIG. 3B shows cumulative release of OVA for PGCLM61, PGCL61 and crosslinked PGCLM61 which is denoted NPGCLM61, obtained in the working example.

### DETAILED DESCRIPTION

We turn now to the method of the first embodiment.

The unsaturated functionalized polyhydric alcohol esters of polyesters for step (a) include the double bond functionalized polyhydric alcohol esters of polyesters described in U.S. Pat. No. 6,592,895 and Lang, M., et al., Journal of Polymer Science: Part A: Polymer Chemistry, Vol. 40, 1127-1141 (2002) and can be synthesized as indicated therein.

Very preferred double bond functionalized polyhydric alcohol esters of polyesters are obtained by polymerizing s-caprolactone monomer in the presence of glycerol to provide esters with free hydroxyl at terminal ends of the acyl groups (PGCL) and reacting with maleic anhydride to convert some or each of the free hydroxyls to moiety containing 2-carboxy ethenyl group, particularly to 1-carboxyl-2-carboxy ethenyl. These double bond functionalized polyhydric alcohol esters of polyesters and their preparation are described in U.S. Pat. No. 6,592,895 and may be referred to herein as PGCLM. The resulting compounds have number average molecular weight, Mₙ, ranging for example, from 1,000 to 50,000.

The solvent for step (a) is one that dissolves the unsaturated functionalized polyhydric alcohol ester of polyester at room temperature and which has a boiling point ranging, for example, from 30-45° C. (which allows for easy removal of solvent). A preferred solvent for step (a) is dichloromethane (bp of 38.9-40° C.). Other suitable solvents for step (a) include chloroform, ethyl acetate, and N,N-dimethylformamide.

For step (a), the double bond functionalized polyhydric alcohol esters of polyesters are dissolved in the hydrophobic organic solvent in an amount ranging, for example, from 0.5 to 10% w/v. An increase in concentration causes increase in mean diameter of microsphere ultimately obtained as well as in loading efficiency of water soluble drug loaded as described hereinafter and at least up to 6% w/v causes increase in loading level (drug %, w/w of microsphere).

We turn now to the drug and/or other biologically active agent loaded into the microspheres for sustained release therefrom; i.e., the drug and/or other biologically active agent. The drug or biologically active agent can be, for example, carrier of aminoxyl radical or an anti-inflammatory agent (e.g., serolimos) or antiproliferative drug (e.g., paclitaxel), or biologic, or protein, or cytokine, or oligonucleotide including antisense oligonucleotide, or gene, or carbohydrate, hormone, or as described above.

For step (b), water-soluble drug and/or other biologically active agent is dissolved in water at a level of 1-500 mg per ml.

The volume ratio of solution of step (b) to solution of step (a) admixed in step (c) can range, for example, from 3:1 to 10:1, e.g., a volume ratio of water to dichloromethane in step (c) ranging from 9:1 to 1:1. Admixing can be carried out at 800 to 1,000 rpm for 5 minutes to 1 hour using a magnetic stirrer.

The stabilizer for step (d) is a compound which is insoluble in the solvent of step (a), is removable by washing with water and is stable in sunlight and artificial light and reduces the interfacial tension between aqueous and organic phases and limits collapse of droplets in step (e) before hardened microspheres are obtained in step (f).

A preferred stabilizer is polyvinyl alcohol (PVA) having a number average molecular weight ranging from 10,000 to 30,000 which is 85-90% hydrolyzed and is present in the solution formed in step (d) in amount ranging from 0.5 to 10%, e.g., 0.5 to 5%, w/v. Use of the PVA in an amount less than 0.5% w/v results in coagulation of microspheres and subsequent formation of large aggregates which is undesirable.

Substitutes for the PVA include Pluronic F68 (ethylene oxide/propylene oxide block copolymer having the structure:
HO(C₂H₄₀)ₐ(C₃H₆O)_{b}(C₂H₄O)ₐH
where a is 80 and b is 27, having molecular weight ranging from 7680 to 9510 and CAS Registry Number 9003-11-6), human serum albumin (HSA), and sodium chlorate.

The volume of solution of step (d) to emulsion of step (c) admixed in step (e) can range, for example, from 5:1 to 1:1.

The evaporation of step (f) is readily carried out with stirring while exposing the emulsion formed in step (e) to the atmosphere while maintaining the emulsion at room temperature to 45° C. Upon evaporation the microspheres precipitate and become hardened because of the greater presence of stabilizer at the surface of emulsion droplets.

The recovery of step (g) may be carried out by centrifuging to collect the microspheres, washing the microspheres with distilled water to remove PVA or other stabilizer and freeze drying and then storing until used.

We turn now to the case where the surface of a microsphere is converted to a hydrogel. This is effected by including a photoinitiator in the solution formed in step (a), e.g., at a level of 0.05 to 0.5% w/w of the double bond functionalized polyhydric alcohol ester, e.g., 2,2-dimethoxy 2-phenyl acetophenone (DMPA) at a level of 0.1% (w/w of PGCLM), and then admixing the solutions formed in step (a) and step (b) to form emulsion of step (c) and, in step (e) admixing solution formed in step (d) with emulsion formed in step (c) and in carrying out step (e) causing cross linking at double bond functionality, e.g., by photocrosslinking, i.e., causing vinyl bonds to break and form cross-links by the application of radiant energy, e.g., by irradiating with a long wavelength UV lamp (365 nm, 16 watt) at room temperature while gently stirring overnight. After that, the same hardened microsphere formation and collection procedures can be used, as when hydrogel is not formed at microsphere surface.

We turn now to the third embodiment. The injectable microspheres in the working example hereinafter had a mean transverse dimension ranging from about 20 μm to about 55 mμ and were loaded with from about 1 to about 8 percent by weight of the microsphere of drug or other biologically active agent. In one alternative, the surface of a PGCLM microsphere is converted to a hydrogel as described above. In all cases double bonds and carboxyl groups at the surface of the microspheres can be reacted to covalently bond to drug or other biologically active agent. Loading efficiencies (actual drug loaded (g)/theoretical load (g)×100%) are readily obtained up to about 45% (46% was obtained in one case), loading levels (actual loaded drug (g)/microsphere weight (g)×100%) are readily obtained up to about 8% and cumulative release in 0.1M phosphate buffered saline (PBS) at 37° is obtained up to about 50% over 50 days.

As indicated above, the injectable microspheres of the third embodiment including those where hydrogel is formed at microsphere surface, are biodegradable.

In the expression “some or each”, “some” means more than one and less than all, and “each” connotes all.

The invention is illustrated by the following working example.

### WORKING EXAMPLE

The polymer used was the PGCL-Ma-3 described in U.S. Pat. No. 6,592,895 and was made up as described in U.S. Pat. No. 6,592,895.

The procedure for polymer preparation was as follows:

In brief, hydroxyl functionalized three-arm poly (ε-caprolactone) (PGCL) was synthesized by ring-opening polymerization of ε-caprolactone (CL) in the presence of glycerol, which acted as a core, at the 20:1 feed molar ratio of CL to the hydroxyl group of glycerol and stannous octoate catalyst (0.1 wt % of CL) in a silinized Pyrox press reaction tube. After being vacuumed and refilled with dry argon several times, the polymerization tube was sealed in vacuum and placed in an oil bath at 130° C. for 48 hours. The polymer obtained (PGCL) was dissolved in chloroform and then gently poured into excess petroleum ether to precipitate the product. The precipitates were washed with distilled water four times and dried over P₂O₅ in vacuum at room temperature until a constant weight was obtained.

For the synthesis of double-bond-functionalized three-arm poly (ε-caprolactone maleic acid (PGCLM), PGCL and 5 equiv of the hydroxyl functionality of maleic anhydride were placed in a three-necked flask under a dry N₂ environment and the flask was heated to 130° C. for one day. The reaction mixture was then cooled to room temperature and dissolved in chloroform. This chloroform solution was poured into excess petroleum ether to precipitate PGCLM. The powder precipitate was stirred in 500 mL of distilled water for 4 hours for the removal of any excess maleic anhydride. After filtration, the precipitate washed with distilled water four times and dried over P₂O₅ in vacuum at room temperature until a constant weight was obtained.

Linear high molecular weight (poly (ε-caprolactone), Mn=56.9 kg/mol), was also synthesized by the same method but without glycerol or any other alcohol as an initiator, and the amount of stannous octoate was decreased to 0.05 wt % CL. This high molecular weight PCL was used as control for PGCL and PGCLM characterization, microspheres preparation and protein encapsulation.

In another case cross-linked PGCLM (NPGCLM) was made up by including 0.1% (w/w) DMPA in the reaction mixture for forming PGCLM and after forming PGCLM, then irradiating with a long wavelength lamp (365 nm, 16 watts) to cause crosslinking at double bonds.

In another case 2,2′-bis (2-oxazolime) linked poly (ε-caprolactone) (PCL-O); described in Tarvainen, T., et al., J. Control. Release, 86 (2-3), 213-222 (2003) was obtained.

The physiochemical characteristics of the PGCL, PGCLM, NPGCLM, PCL and PCL-O mentioned above are set forth in Table 1 below:

TABLE 1

| | | | | | Degree of |
|---|---|---|---|---|---|
| | Mn | T_{g} | Tₘ | ΔHₘ | Crystal- |
| Polymer | (kg/mol)^{a} | (° C.) | (° C.) | (J/g) | linity (%)^{b} |
| PGCL^{c} | 15.4 | — | 52.5 | 61.3 | 45.4 |
| PGCLM^{d} | 13.3 | — | 48.9 | 64.8 | 48.0 |
| NPGCLM | 13.3 | — | 51.3 | 47.3 | 35.0 |
| PCL | 56.9 | −59 | 59 | — | 52 |
| PCL-O | 39.1 | −54 | 52 | — | 47 |

^{a}Determined by GPC with polystyrene standards;
^{b}Degree of Crystallinity = (ΔH_{m, sample}/H°_{m, 100% crystalline}) × 100%;
^{c}The molar ratio of CL monomer to hydroxyl of glycerol is 20/1;
^{d}PGCL with 5 equiv of maleic anhydride as feed molar ratio.

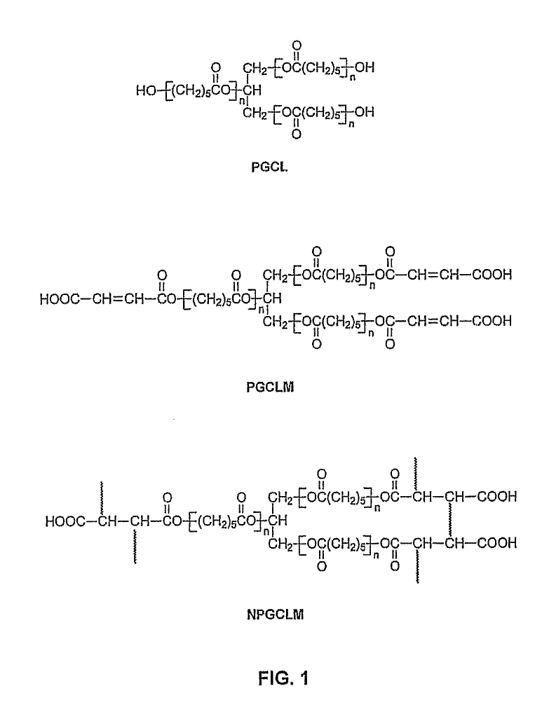

FIG. 1 shows chemical structures for PGCL and PGCLM and a representative structure for NPGCLM.

PGCLM and NPGCLM microspheres without drug loading were prepared.

For the microspheres without drug loading, PGCL-Ma-3 was dissolved in dichloromethane (4%, 6%, 8% w/v). The solutions were emulsified in 50 mL aqueous 1% (w/v) polyvinyl alcohol (PVA) (molecular weight of 12,000-23,000 and 87-89% hydrolyzed) by admixing and stirring for 30 minutes at 900 rpm. The resulting solution was stirred at room temperature (22° C.) by a magnetic stirrer overnight to evaporate the dichloromethane. Samples were collected by centrifugation (800 rpm for 6 hours) at 22° C. and washed with distilled water at least four times to remove the PVA. The samples were freeze dried for three days in a Virtis Freeze Drier under vacuum at −45° C. to obtain microsphere products which were stored in vacuum desiccators.

To prepare microspheres having crosslinked surface network (hydrogel) structure, DMPA at a level of 0.1% (w/w) of the PGCLM was added to the PGCLM solution which in turn was emulsified to form a water-in-oil-in-water emulsion. The emulsion was then irradiated with a long wavelength UV lamp (365 nm, 16 watt) at room temperature to cause surface crosslinking and gently stirred overnight at room temperature to evaporate the dichloromethane. Collection procedures for the resulting microspheres were the same as in the above paragraph.

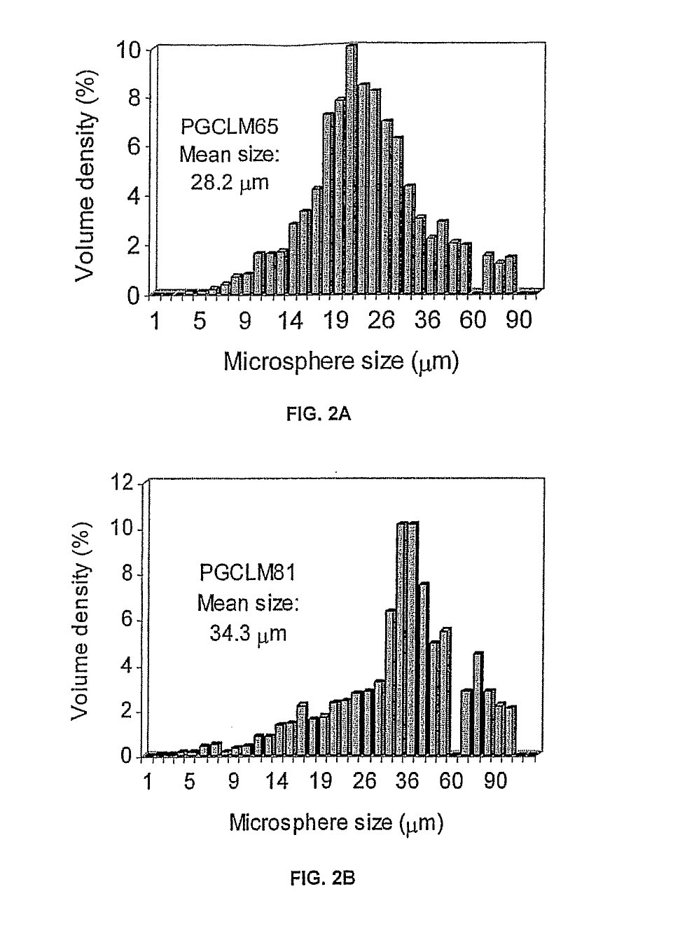

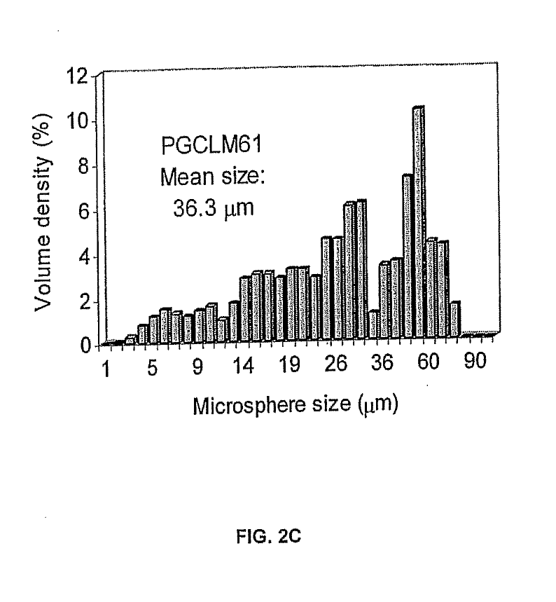

FIGS. 2A, 2B, and 2C show size distribution respectively for PGCLM65 microspheres, PGCLM81 microspheres and PGCLM61 microspheres, that were obtained.

For drug loading, an ovalbumin protein (albumin, chicken egg, Grade V), denoted OVA, was selected to represent drug to be loaded. It has been used as an antigen in inducing antibody cell-mediated immune responses as well as for oral vaccine delivery.

PGCLM and NPGCLM microspheres loaded with OVA were prepared by a water-in-oil-in-water (w/o/w) emulsion technique.

For OVA loaded PGCLM microspheres, loading was carried out as follows:

1 mL OVA aqueous solutions (containing 40, 80 or 170 mg OVA) were dispersed in 10 mL of PGCLM solution (4%, 6%, 8% w/v in dichloromethane) with vigorous stirring (900 rpm for 15 minutes with a magnetic stirrer) to form a water-in-oil emulsion where aqueous OVA solution was the disperse phase in PGCLM solution continuous phase. The resulting w/o emulsion was emulsified in 50 mL aqueous 1% PVA(Mₙ=12,000-23,009 and 87-89% hydrolyzed) solution (w/v) by mixing for 30 minutes at 900 rpm with a magnetic stirrer to form a w/o/w emulsion. The resulting w/o/w emulsion was gently stirred overnight at room temperature (22° C.) by a magnetic stirrer (EYELA Magnetic Stirrer RC-2) to evaporate organic solvent leaving hardened microspheres loaded with OVA, undissolved in the aqueous continuous phase. The microspheres were collected by centrifugation at 22° C. (International Centrifuge, Clinical Model, International Equipment Co., Needham Hts, Mass. 02194 USA) and washed with distilled water at least four times to remove PVA emulsifier. The sample was then freeze-dried for 3 days in a Virtis Freeze Drier (Gardiner, N.Y.) under vacuum at 45° C. to obtain the microspheres which were stored in vacuum desiccators at 40° C. before characterization and use.

In another case, the procedure was the same as above but DMPA at 0.1% (w/w of PGCLM) was added to the solution of PGCLM before it was used to form w/o emulsion with aqueous solution of OVA whereupon the w/o emulsion was admixed with the PVA aqueous solution to form a w/o/w emulsion which was irradiated by using a long wavelength UV lamp (365 nm, 16 watts) at room temperature with gentle stirring overnight. After that, the same procedure as used above, was used to collect the microspheres. The result was cross-linked surface network structure microspheres denoted NPGCLM, loaded with OVA.

Characteristics of OVA-loaded PGCLM and NPGCLM microspheres are shown in Table 2 below.

TABLE 2

| | Polymer^{a} | OVA con. | PVA^{b} | DCM/H₂O | Mean Diam. | OVA loading^{c} | Loading |
|---|---|---|---|---|---|---|---|
| Code | (% w/v) | (mg) | (%, w/v) | (v/v) | (dᵥₛ, μm) | (%, w/w of MS) | Effic. (%)^{d} |
| PGCLM41 | 4 | 40 | 1 | 1/20 | 51.1 | 6.0 | 41.1 |
| PGCLM61 | 6 | 40 | 1 | 1/20 | 36.3 | 7.6 | 42.2 |
| PGCLM81 | 8 | 40 | 1 | 1/20 | 34.3 | 4.1 | 43.2 |
| PGCL61 | 8 | 40 | 1 | 1/20 | 34.2 | 7.2 | 36.2 |
| NPGCLM61 | 8 | 40 | 1 | 1/20 | 27.6 | 4.2 | 45.3 |

^{a}Dichloromethane (DCM) as solvent, PGCL Mₙ = 15.4 kDa; PGCLM Mn = 13.3 kDa; NPGCLM Mn = 13.3 kDa;
^{b}Water as solvent;
^{c}OVA loading (%) = actually loaded OVA (g)/microspheres weight (g) × 200%;
^{d}Loading efficiency = actually loaded OVA (g)/feed OVA (g) × 100%.

In the above Table 2 and hereinafter, PGCLM41 means microspheres made using 4% w/v PGCLM polymer and 1% w/v PVA, PGCLM61 means PGCLM microspheres made using 6% w/v PGCLM polymer and 1% w/v PVA, PGCLM81 means PGCLM microspheres made using 8% PGCLM w/v polymer and 1% w/v PVA. PGCL61 means PGCL microspheres made using 6% w/v PGCL and 1% w/v PVA. NPGCLM61 means NPGCLM microspheres made using 6% w/v PGCLM polymer and 1% w/v PVA.

Mean diameter was determined according to the following procedure. Dried microsphere powder samples were first suspended in HPLC grade water (5-10% vol.) and then slightly sonicated to obtain a homogeneous suspension. Size measurement was carried out using a laser light scattering method (Brinkman Particle Size Analyzer 2010, Brinkman Instruments, Inc., Westbury, N.Y.).

Table 2 shows that mean diameter depended on PGCLM concentration in DCM/H₂O solvent. An increase in PGCLM concentration from 4% to 8% w/v in a fixed volume of DCM with constant PVA concentration (1% w/v) resulted in decrease in mean microsphere size. The crosslinked NPGCLM had smaller mean diameter than the uncrosslinked PGCLM.

The loading efficiency data in Table 2 illustrates that there is a relationship between polymer concentration and drug loading. The OVA loading efficiency for PGCLM and NPGCLM microspheres ranged from 41% to 45% (w/w). The corresponding loading levels ranged from 4.1 to 7.6% (w/w). Table 2 indicates that an increase in polymer concentration at constant OVA and PVA concentration led to a slight increase in loading efficiency and that the photocrosslinked case improved loading efficiency despite the fact that the mean diameter was less.

Influence of ovalbumin (OVA) concentration on OVA loading efficiency at constant microsphere formulation (fixed polymer concentration and its ratio to PVA stabilizer) is shown in Table 3 below.

TABLE 3

| | Polymer^{a} | OVA con. | PVA^{b} | DCM/H₂O | Mean Diam. | OVA loading^{c} | Loading |
|---|---|---|---|---|---|---|---|
| Code | (%, w/v) | (mg) | (%, w/v) | (v/v) | (dᵥₛ, μm) | (%, w/w of MS) | Effic. (%)^{d} |
| PGCLM61-1 | 6 | 10 | 1 | 1/20 | 35.2 | 1.4 | 43.0 |
| PGCLM61-2 | 6 | 40 | 1 | 1/20 | 36.3 | 7.6 | 42.2 |
| PGCLM61-3 | 6 | 85 | 1 | 1/20 | 35.7 | 8.1 | 28.7 |

^{a}Dichloromethane as solvent, PGCLM Mn = 13.3 kDa;
^{b}Water as solvent;
^{c}OVA Loading (%) = actually loaded OVA (g)/microspheres weight (g) × 100%;
^{d}Loading efficiency = actually loaded OVA (g)/feed OVA (g) × 100%.

In the above Table 3, PGCLM61 means microspheres made using 6% w/v PGCLM polymer and 1% w/v PVA. The data show that an increase in the OVA concentration in the aqueous phase (from 10 to 40 to 85 mg) resulted in a reduction of OVA loading efficiency from 43% to 28% without a significant change in microsphere mean diameter.

The influence of PVA concentration (ranging from 0.5% w/v to 5% w/v) on OVA loading efficiencies in microspheres formed from 6% and 8% PGCLM concentration is shown in Table 4 below.

TABLE 4

| | Polymer^{a} | OVA con. | PVA^{b} | DCM/H₂O | Mean Diam. | OVA loading^{c} | Loading |
|---|---|---|---|---|---|---|---|
| Code | (%, w/v) | (mg) | (%, w/v) | (v/v) | (dᵥₛ, μm) | (%, w/w of MS) | Effic. (%)^{d} |
| PGCLM605 | 6 | 40 | 0.5 | 1/20 | 49.3 | 5.4 | 28.7 |
| PGCLM61 | 6 | 40 | 1 | 1/20 | 36.3 | 7.6 | 42.2 |
| PGCLM65 | 6 | 40 | 5 | 1/20 | 28.2 | 7.8 | 45.0 |
| PGCLM805 | 8 | 40 | 0.5 | 1/20 | 53.7 | 3.3 | 34.2 |
| PGCLM81 | 8 | 40 | 1 | 1/20 | 34.3 | 4.1 | 43.2 |
| PGCLM85 | 8 | 40 | 5 | 1/20 | 21.9 | 4.9 | 46.0 |

^{a}Dichloromethane as solvent, PGCLM Mn = 13.3 kDa;
^{b}Water as solvent;
^{c}OVA Loading (%) = actually loaded OVA (g)/microspheres weight (g) × 100%;
^{d}Loading efficiency = actually loaded OVA (g)/feed OVA (g) × 100%.

The PGCLM61 and PGCLM81 of Table 4 are as described for Table 2. For Table 4 and hereinafter, PGCLM605 means microspheres made using 6% (w/v) PGCLM polymer and 0.5% (w/v) PVA; PGCLM65 means microspheres made using 6% (w/v) PGCLM polymer and 5% (w/v) PVA; PGCLM805 means microspheres made using 8% PGCLM (w/v) and 0.5% (w/v) PVA; and PGCLM85 means microspheres made using 8% (w/v) PGCLM and 5% (w/v) PVA.

In the above Table 4, the data shows that for both 6% PGCLM and 8% PGCLM, a lower PVA stabilizer concentration (0.5%) resulted in a lower OVA loading level and loading efficiency and that the higher the PVA stabilizer concentration (1%, 5%) the higher the loading level and loading efficiency and that an increase in PVA concentration caused size reduction in the microspheres.

Scanning electron microscopy (SEM) of surface of the OVA-loaded PGCLM microspheres indicated near spherical, non-aggregated, smooth surface, non-porous appearance surface microspheres.

SEM images of microspheres upon exposure to phosphate buffered saline (PBS) of pH7.4 at 37° C. showed small pores extending from a smooth surface to the interior developing over time and merging with neighboring pores to form larger pores over time. In the same testing, NPGCLM microspheres showed a rough surface with more interconnected and irregular pores attributed to the swollen nature of hydrogel at NPGCLM surface.

The presence of unsaturated functional bonds (>C═C<) on the surface of PGCLM microspheres was confirmed by backscattered electron images (BSE) and X-ray elemental analysis spectra.

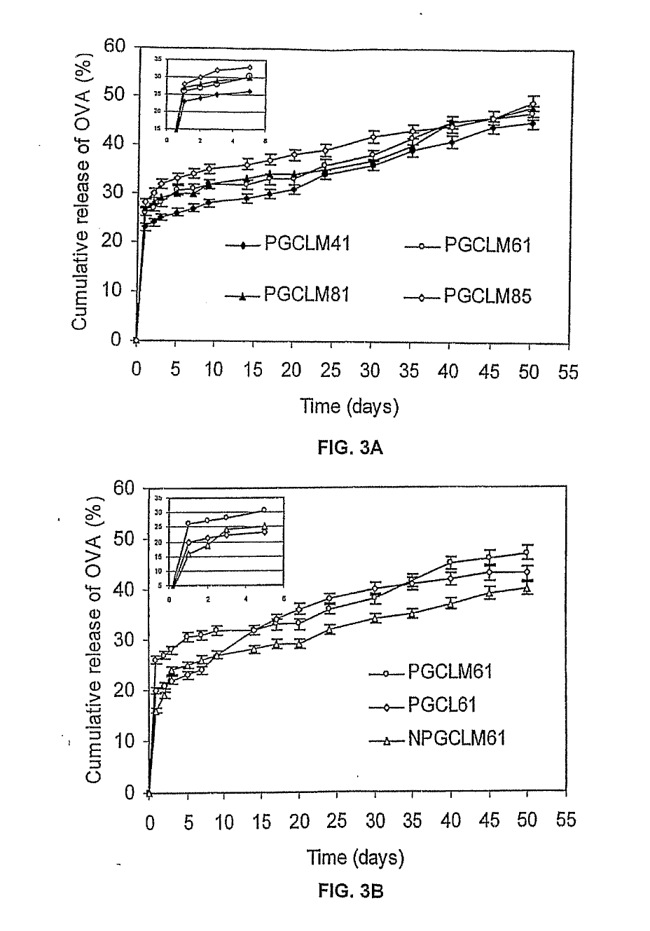

In vitro experiments determining cumulative release of OVA from PGCLM microspheres in PBS at 37° C. showed that regardless of microsphere formation conditions, the OVA release was characterized by an initial burst release followed by a slow and gradual release. Results are shown in FIG. 3A. As shown in FIG. 3A, for the OVA loaded PGCLM microspheres, the one day initial burst cumulative release % was 23, 26, 27, and 28% for PGCLM41, PGCLM61, PGCLM81, and PGCLM85, respectively. At the end of 10 days, the corresponding cumulative releases of OVA % were 28, 32, 32, and 35%. After that period, the release of OVA was slow. At the end of 50 days, the cumulative release of OVA from PGCLM microspheres reaches to 45-49%, in which the PGCLM85 had the highest % OVA released. The release profiles observed indicate that those PGCLM microspheres having smaller size and lower loading efficiency show faster burst release and higher overall cumulative release percentage.

The cumulative release profiles of PGCL61, PGCLM61 and NPGCLM61 microspheres were compared. Results are shown in FIG. 3B. The PGCL61 microspheres differed from PGCLM61 in terms of the lack of maleic monoester chain ends in PGCL61; while PGCLM61 and NPGCLM61 differed from each other in that the NPGCLM61 microspheres were networked (i.e., photocrosslinked) and PGCLM61 microspheres were not. The initial burst releases of OVA were 20, 26 and 16% for PGCL61, PGCLM61 and NPGCLM61 microspheres, respectively. There was a 40% reduction in OVA burst release in NPGCLM61 microspheres compared to the non-networked PGCLM61 microspheres. Thus, the presence of network structure in microspheres reduced the magnitude of initial burst release. At the end of 50 day study period, 43, 47 and 40% cumulative OVA releases were observed for PGCL61, PGCLM61 and NPGCLM61 microspheres, respectively. Thus, the extent of OVA release was in the order of PGCLM61>PGCL61>NPGCLM61.

Gel electrophoresis data showed no fragmentation of OVA by polymer despite formulation and in vitro release testing. The released OVA was shown to have the same molecular weight as fresh OVA samples.

In the experiments herein concentrations below 0.5% w/v for PVA were unable to prevent coagulation of microspheres and subsequent formation of large aggregates, and microspheres made with less than 4% PGCLM were incompletely formed and OVA therein incompletely encapsulated.

The faster release of OVA from PGCLM microspheres compared to PGCL microspheres and NPGCLM microspheres was attributed to the higher hydrophilicity of the PGCLM because of the terminal —COOH groups of the PCL arms without crosslinked network to restrict level of hydration and protein release.

The long sustained release of OVA from the microspheres was sufficient to indicate a continuous delivery of drug without cyclic variation in drug concentration with time thereby offering good pharmacological efficiency over an extended time.

Variations

The foregoing description of the invention has been presented describing certain operable and preferred embodiments. It is not intended that the invention should be so limited since variations and modifications thereof will be obvious to those skilled in the art, all of which are within the spirit and scope of the invention.

1. A method for forming a drug/biologically active agent laden injectable microsphere comprising the steps of:
(a) dissolving unsaturated functionalized polyhydric alcohol ester of polyester in hydrophobic organic solvent,
(b) dissolving drug and/or other biologically active agent in water,
(c) admixing the solutions formed in step (a) and step (b) to form an emulsion where the solution formed in step (a) constitutes the continuous phase and the solution formed in step (b) constitutes the disperse phase,
(d) dissolving stabilizer in water,
(e) admixing the solution formed in step (d) with the emulsion formed in step (c) to form a water-in-oil-in-water emulsion where solution of step (d) constitutes the continuous phase and the emulsion formed in step (c) constitutes the disperse phase,
(f) evaporating the organic solvent from the water-in-oil-in-water emulsion formed in step (e), to form hardened microspheres from the unsaturated functionalized polyhydric alcohol ester of polyester, encapsulating said drug and/or other biologically active agent therein,
(g) recovering drug/biologically active agent laden hardened microsphere.

2. The method of claim 1 where the unsaturated functionalized polyhydric alcohol ester of polyester is obtained by polymerizing ε-caprolactone monomer or a blend of ε-caprolactone monomer and lactide monomer or glycolide monomer in the presence of polyhydric alcohol containing from 3 to 6 hydroxyl groups to form polyhydric alcohol ester of polyester where the acyl groups contain free hydroxyl at their terminal ends and reacting with maleic anhydride to convert some or each of the free hydroxyls to moiety containing 1-carboxyl-2-carboxy ethenyl group.

3. The method of claim 2 where the double bond functionalized polyhydric alcohol ester of polyester is obtained by polymerizing ε-caprolactone monomer in the presence of glycerol to form the polyhydric alcohol ester of polyester where the acyl groups contain free hydroxyl at their terminal ends and reacting with maleic anhydride to convert some or each of the free hydroxyls to moiety containing 1-carboxyl-2-carboxy ethenyl groups.

4. The method of claims 3 where the double bond functionalized polyhydric alcohol ester of polyester has a number average molecular weight, Mₙ, ranging from 1,000 to 50,000.

5. The method of claim 4 where the solvent of step (a) is one that dissolves the unsaturated functionalized polyhydric alcohol ester of polyester at room temperature and has a boiling point ranging from 30-45° C.

6. The method of claim 5 wherein the solvent is dichloromethane.

7. The method of claim 4 where the stabilizer is soluble in water at a stabilizing effective concentration and is insoluble in the solvent of step (a) and is removable by washing with water and is stable in sunlight and artificial light.

8. The method of claim 7 where the stabilizer is polyvinyl alcohol having a number average molecular weight ranging from 10,000 to 30,000 and is 85-90% hydrolyzed and is present in the solution formed in step (d) in an amount ranging from 0.5% to 5% w/v.

9. The method of claim 4 where the volume ratio of solution formed in step (b) to solution formed in step (a) admixed in step (c) ranges from 3:1 to 10:1.

10. The method of claim 4 where photoinitiator is included in the solution formed in step (a) and the admixture formed in step (e) is irradiated to obtain photocrosslinking to provide hydrogel surface on disperse phase particles.

11. A method for forming a drug/biologically active agent laden injectable microsphere comprising the steps of:
(a) dissolving unsaturated functionalized polyhydric alcohol ester of polyester in hydrophobic organic solvent,
(b) dissolving drug and/or other biologically active agent in water,
(c) admixing the solutions formed in step (a) and step (b) to form an emulsion where the solution formed in step (a) constitutes the continuous phase and the solution formed in step (b) constitutes the disperse phase,
(d) dissolving stabilizer in water,
(e) admixing the solution formed in step (d) with the emulsion formed in step (c) to form a water-in-oil-in-water emulsion where solution of step (d) constitutes the continuous phase and the emulsion formed in step (c) constitutes the disperse phase,
(f) evaporating the organic solvent from the water-in-oil-in-water emulsion to form hardened microsphere with drug/biologically active agent covalently bonded to unsaturated functionality of polyhydric alcohol ester of polyester forming the microsphere,
(g) recovering drug/biologically active agent laden hardened microsphere.

12. Injectable microsphere having a mean transverse dimension ranging from 10 to 60 μm, formed of hardened unsaturated functionalized polyhydric alcohol ester of polyester loaded with from 1 to 10% by weight of the microsphere of a drug or other biologically active agent for sustained release after injection of the microsphere.

13. The injectable microsphere of claim 12 where the unsaturated functionalized polyhydric alcohol ester of polyester is obtained by polymerizing ε-caprolactone monomer in the presence of glycerol to form polyhydric alcohol ester of polyester where the acyl groups contain free hydroxyl at their terminal ends and reacting with maleic anhydride to convert some or each of the free hydroxyls to 1-carboxyl-2-carboxy ethenyl group.

14. The injectable microsphere of claim 13 where the surface of the microsphere has been converted to a hydrogel.

15. The injectable microsphere of claims 12 where drug/biologically active agent is encapsulated in the microsphere.

16. The injectable microsphere of claim 12 where drug/biologically active agent is covalently bonded to unsaturated functionality of polyhydric alcohol ester of polyesters forming the microsphere.
